(19) United States
(12) Patent Application Publication
(10) Pub. No.: US 2015/0197790 A1
(43) Pub. Date: Jul. 16, 2015

(54) INTERCALATING DYES FOR DIFFERENTIAL DETECTION

(71) Applicant: Bio-Rad Laboratories, Inc., Hercules, CA (US)

(72) Inventor: Svilen Tzonev, Pleasanton, CA (US)

(21) Appl. No.: 14/594,950

(22) Filed: Jan. 12, 2015

Related U.S. Application Data
(60) Provisional application No. 61/926,172, filed on Jan. 10, 2014.

(51) Int. Cl. C12Q 1/68

(52) U.S. Cl. CPC C12Q 1/6809; C12Q 1/6827

(57) ABSTRACT

Methods and compositions are provided for detection and quantification of nucleic acid sequences.

### CROSS-REFERENCE TO RELATED APPLICATION

This application claims priority to U.S. Provisional Application No. 61/926,172, filed on Jan. 10, 2014, the contents of which are hereby incorporated by reference in the entirety for all purposes.

### BACKGROUND OF THE INVENTION

Nucleic acids can be detected or quantified in order to search for useful genes, diagnose diseases or identify organisms. Molecular approaches designed to detect or quantify nucleic acids can be used to detect mutations, detect rare nucleic acids, quantify gene expression, measure RNA stability, and the like. Such molecular approaches can be used to determine the relative proportion of nucleic acids. For example, molecular approaches can be used to determine the abundance of a mutant or polymorphic nucleic acid as compared to the abundance of a wild-type nucleic acid in a sample.

Methods and compositions for detecting or quantifying nucleic acids include digital methods in which a sample is partitioned into a number of mixture partitions and the partitions are assayed for the presence or absence of one or more nucleic acids of interest. In some cases, two or more detection reagents, or probes, are used to detect two or more nucleic acids of interest in the partitions. In such cases, cross reactivity of the probes or other detection reagents can make it difficult to definitively detect or quantify the nucleic acids of interest. For example, cross reactivity of detection reagents can cause particular difficulty when one nucleic acid of interest is more prevalent than another, such as when one nucleic acid represents a wild-type sequence, and another represents a mutation. Cross reactivity can also cause particular difficulty when one nucleic acid of interest is very similar to another.

### BRIEF SUMMARY OF THE INVENTION

In some embodiments, the present invention provides a nucleic acid sequence detection method comprising: providing a sample comprising DNA or RNA nucleic acid; partitioning said sample into a set of mixture partitions; detecting a presence or absence of a target nucleic acid in the partitions using a sequence specific detection reagent; and detecting a presence or absence of double-stranded nucleic acid in the partitions using a non-specific detection reagent, thereby detecting the ratio of target nucleic acid to total nucleic acid in the partitions.

In some aspects, the nucleic acid is amplified before detection. In some cases, the non-specific detection reagent is a labeled nucleoside triphosphate, and the step of detecting the presence or absence of double-stranded nucleic acid comprises washing away unincorporated labeled nucleoside triphosphate after amplification. For example, the non-specific detection reagent is a labeled nucleoside triphosphate that is incorporated during an amplification step into one or more structurally different amplicons, and the step of detecting the presence or absence of double-stranded nucleic acid comprises washing away unincorporated labeled nucleoside triphosphate after amplification, and detecting the incorporated label in the one or more structurally different amplicons.

In some aspects, the non-specific detection reagent is an intercalating dye. For example, the intercalating dye can be selected from the group consisting of EvaGreen, picogreen, ethidium bromide, SYBR Green I, SYBR Gold, Yo-Yo, Yo-Pro, TOTO, BOXTO, and BEBO. In some aspects, the sequence specific detection reagent is selected from the group consisting of a structured probe and a linear probe. In some cases, the structured probe is selected from the group consisting of a molecular beacon and a scorpion probe. In some cases, the linear probe is selected from the group consisting of a hybridization probe and a hydrolysis probe.

In some aspects, the non-specific detection reagent is a primer (or mixture of primers, such as a mixture of random primers) that detects total double-stranded nucleic acid.

In one aspect of any one of the preceding embodiments, aspects, or cases, the nucleic acid is RNA, and the method further comprises reverse transcribing the RNA nucleic acid.

In one aspect of any one of the preceding embodiments, aspects, or cases, the method comprises amplifying two or more potential amplicons. In some cases, one of the potential amplicons is present in less than 50%, 40%, 30%, 20%, 10%, 5%, 1%, 0.5%, 0.1%, 0.05%, 0.01%, or fewer of the mixture partitions in which double-stranded nucleic acid is present. In some cases, the sequence specific detection reagent detects one specific amplicon, and the non sequence specific detection reagent detects any amplicon.

In one aspect of any one of the preceding embodiments, aspects, or cases, the sequence specific detection reagent detects a sequence variant. In some cases, the sequence variant is a rare sequence variant. In some cases, the double-stranded nucleic acid is present in a plurality of mixture partitions, and the rare sequence variant is present in less than about 50%, 40%, 30%, 20%, 10%, 5%, 1%, 0.5%, 0.1%, 0.05%, 0.01%, or fewer mixture partitions.

In one aspect of any one of the preceding embodiments, aspects, or cases, the method comprises determining a total nucleic acid concentration by counting the number of mixture partitions in which non-specific detection reagent detects nucleic acid, or detects amplified nucleic acid. In some cases, the method further comprising determining a target nucleic acid sequence concentration by counting the number of mixture partitions in which sequence specific detection reagent detects nucleic acid, or detects amplified nucleic acid. In some cases, the method further comprises determining a ratio of mixture partitions in which the sequence specific detection reagent detects nucleic acid (e.g., amplified nucleic acid) to mixture partitions in which the non sequence specific detection reagent detects nucleic acid (e.g., amplified nucleic acid), wherein the ratio represents the proportion of nucleic acids in the sample that comprise the target nucleic acid. In some cases, the method further comprises reporting the ratio.

In one embodiment, the invention provides a nucleic acid sequence detection method comprising: providing a sample comprising DNA or RNA nucleic acid, wherein the DNA or RNA nucleic acid comprises a first target and a second target; partitioning said sample into a set of mixture partitions; and detecting the first target and the second target in at least one mixture partition with a specific detection reagent that binds to the first target (e.g., specifically detects the first target but not the second target) if present, and a nonspecific detection reagent that binds, and thereby detects, both targets if present; thereby determining a concentration of the first target and a concentration of the first and second target in the sample.

In one aspect, the method further comprises amplifying the targets in the mixture partitions; detecting comprises detecting the amplification of the first and second target; and the specific detection reagent binds to amplicons representing the first target (e.g., specifically binds—and thereby detects—amplicons of the first target but not the second target) and the non-specific detection reagent binds to and thereby detects amplicons representing the first and/or the second target.

In one aspect, the detecting comprises determining the presence or absence of the first target and determining the presence or absence of the first or second target in the at least one mixture partition. In some cases, the detecting is performed on a plurality of mixture partitions. In some cases, the method further comprises determining a ratio of mixture partitions comprising the first target to mixture partitions comprising the first or the second target. In some cases, the method further comprises reporting the ratio.

In one aspect, the first target is a mutant or a polymorphism and the second target is a wild-type nucleotide sequence.

In one embodiment, the present invention provides a composition comprising a mixture partition of less than about 100 nL comprising: a nucleic acid comprising DNA or RNA; a non-specific detection reagent; and a sequence specific detection reagent. In one aspect, the composition further comprises amplification reagents. In one aspect, the non-specific detection reagent is selected from the group consisting of EvaGreen, ethidium bromide, SYBR Green, SYBR Gold, Yo-Yo, Yo-Pro, TOTO, BOXTO, and BEBO. The non-specific detection reagent can be a primer that detects total double-stranded nucleic acid. The non-specific detection reagent can be a labeled nucleoside triphosphate. The sequence specific detection reagent can be selected from the group consisting of a molecular beacon, a scorpion probe, a hybridization probe, and a hydrolysis probe.

In one embodiment, the present invention provides a set of mixture partitions, wherein a plurality of the mixture partitions comprises one of the foregoing compositions. In some cases, the set comprises at least about 100, 200, 500, or 1000 mixture partitions. In some cases, the plurality of the mixture partitions comprises double-stranded nucleic acid. In some cases, a majority of the mixture partitions comprising double-stranded nucleic acid do not comprise a target nucleic acid. In some cases, the target nucleic acid is a sequence variant.

### BRIEF DESCRIPTION OF THE DRAWINGS

FIG. 1: Depicts a typical experiment for detecting a rare SNP in a sample containing a population of DNA molecules using digital droplet PCR with two sequence specific probes. Droplets in which neither wild-type nor the rare variant are detected are denoted as “−−” and cluster in the bottom left quadrant. Droplets in which only wild-type nucleic acids are detected are denoted “+−” and cluster in the bottom right. Droplets in which both wild-type and the rare variant are detected are denoted “++” and cluster in the top right. Droplets in which only the rare variant are detected are denoted “−+” and cluster in the top left.

FIG. 2: Depicts an experiment for detecting a rare SNP in a sample containing a population of DNA molecules using digital droplet PCR with one sequence specific probe and one non-specific probe. Droplets in which neither wild-type nor the rare variant are detected are denoted as “−−” and cluster in the bottom left quadrant. Droplets in which only wild-type nucleic acids are detected are denoted “+−” and cluster in the bottom right. Droplets in which both wild-type and the rare variant are detected are denoted “++” and cluster in the top right. Droplets in which only the rare variant are detected are denoted “−+” and also cluster in the top right.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Spring Harbor Lab Press (Cold Spring Harbor, N.Y. 1989). The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

As used herein, the term “partitioning” or “partitioned” refers to separating a sample into a plurality of portions, or “partitions.” Partitions can be solid or fluid. In some embodiments, a partition is a solid partition, e.g., a micro or nano channel. In some embodiments, a partition is a fluid partition, e.g., a droplet. In some embodiments, a fluid partition (e.g., a droplet) is a mixture of immiscible fluids (e.g., water and oil), or an emulsion. In some embodiments, a fluid partition (e.g., a droplet) is an aqueous droplet that is surrounded by an immiscible carrier fluid (e.g., oil). In other embodiments, a fluid partition is an aqueous droplet that is physically or chemically separated from adjacent aqueous droplets such that nucleic acid, buffers, salts, or other molecules in one droplet do not diffuse into adjacent droplets.

The term “detection reagent” refers to a molecule (e.g., a dye, protein, nucleic acid, aptamer, etc.) that interacts with or binds to a target molecule such as a nucleic acid. Non-limiting examples of molecules that interact with or bind to a target molecule include dyes (e.g., intercalating dyes), nucleic acids (e.g., oligonucleotides), proteins (e.g., antibodies, transcription factors, zinc finger proteins, non-antibody protein scaffolds, etc.), and aptamers.

The term “sequence specific detection reagent” refers to a molecule (e.g., a nucleic acid, a protein, an aptamer, etc.) that specifically binds to a particular sequence or otherwise specifically detects a particular sequence. In some embodiments, sequence specific detection reagents can exhibit cross reactivity with non target nucleic acids.

The term “specifically binds to” or “specifically interacts with” refers to a detection reagent (e.g., an oligonucleotide, an aptamer, or an antibody) that binds to a target sequence with at least 2-fold greater affinity than one or more non-target sequences, e.g., at least 4-fold, 5-fold, 6-fold, 7-fold, 8-fold, 9-fold, 10-fold, 20-fold, 25-fold, 50-fold, 100-fold, or 1000-fold or greater affinity. As used herein, a greater affinity can be measured, for example, as a lower dissociation constant (K_{d}). For example, a detection reagent that specifically binds a particular target nucleic acid will typically bind the target nucleic acid with at least a 10-fold greater affinity than one or more non-target nucleic acids (e.g., a K_{d} that is 1/10^{th} the K_{d} for a non-target nucleic acid). In some cases, the non-target nucleic acid includes nucleic acids that are substantially similar to the target nucleic acid. For example, in some cases, the non-target nucleic acid includes nucleic acids that differ by about one nucleotide (e.g., differ by 1, 2, 3, 4, 5, 6, 7, 8, 9, or 10 nucleotides) from the target nucleic acid. As another example, the non-target nucleic acid can include a conserved region or domain that is substantially similar to the target nucleic acid and other regions or domains that are substantially different. In some cases, the non-target nucleic acid includes a nucleic acid that is substantially different from the target nucleic acid. In some cases, a detection reagent that specifically binds a particular target nucleic acid will hybridize to the target nucleic acid with a melting temperature that is at least 0.5, 1, 2, 5, 10, 15, 20, or 25° C. higher than the melting temperature when hybridized to a non target nucleic acid.

In some contexts, “specifically binds to” or “specifically interacts with” refers to a detection reagent (e.g., an oligonucleotide, an aptamer, or an antibody) that binds to and detects a target sequence, but does not substantially bind to or detect a non-target sequence in a complex mixture. For example, a detection reagent that specifically binds to a target sequence might not detect, or not substantially detect, non-target nucleic acids present in a cell lysate or a nucleic acid preparation comprising the genome or transcriptome of an organism or sample.

The term “non-specific detection reagent” refers to a molecule that binds to or detects nucleic acids in general (e.g., total nucleic acid, total amplified nucleic acid, total reverse transcribed nucleic acid, total DNA, or total double stranded nucleic acid). For example, a non-specific detection reagent can include a dye, such as an intercalating dye, that binds nucleic acid. Alternatively, a non-specific detection reagent can include a nucleotide that binds to or detects a universal sequence incorporated into a nucleic acid. For example, nucleic acids in a sample may be amplified by one or more primers containing a detectable sequence. The non-specific detection reagent can detect the detectable sequence thus incorporated during the amplification reaction. In some cases, the non-specific detection reagent can distinguish between amplified and non-amplified nucleic acid. In other cases, the non-specific detection reagent can distinguish between single stranded and double stranded nucleic acid. In some cases, the non-specific detection reagent can distinguish between DNA and RNA. In some cases, the non-specific detection reagent fluoresces or increases in fluorescence when bound to nucleic acid.

The terms “label” and “detectable label” interchangeably refer to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes, biotin, digoxigenin, or haptens and proteins, nucleic acids, or other entities which can be made detectable, e.g., by incorporating a radiolabel into an oligonucleotide, peptide, or antibody specifically reactive with a target molecule. Any method known in the art for conjugating an oligonucleotide to the label can be employed, e.g., using methods described in Hermanson, Bioconjugate Techniques 1996, Academic Press, Inc., San Diego.

A molecule that is “linked” to a label (e.g., as for a labeled probe as described herein) is one that is bound, either covalently, through a linker or a chemical bond, or noncovalently, through ionic, van der Waals, electrostatic, or hydrogen bonds to a label such that the presence of the molecule can be detected by detecting the presence of the label bound to the molecule.

“Intercalating dye” refers to molecules that intercalate double stranded nucleic acid, such as double stranded DNA. In some embodiments, intercalating dyes fluoresce. In some cases, intercalating dyes increase in fluorescence when bound to nucleic acid as compared to their fluorescence when free in solution. Numerous intercalating dyes are known in the art. Some non-limiting examples include 9-aminoacridine, ethidium bromide, a phenanthridine dye, EvaGreen, PICO GREEN (P-7581, Molecular Probes), EB (E-8751, Sigma), propidium iodide (P-4170, Sigma), Acridine orange (A-6014, Sigma), thiazole orange, oxazole yellow, 7-aminoactinomycin D (A-1310, Molecular Probes), cyanine dyes (e.g., TOTO, YOYO, BOBO, and POPO), SYTO, SYBR Green I (U.S. Pat. No. 5,436,134: N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR Green II (U.S. Pat. No. 5,658,751), SYBR DX, OliGreen, CyQuant GR, SYTOX Green, SYTO9, SYTO10, SYTO17, SYBR14, FUN-1, DEAD Red, Hexidium Iodide, ethidium bromide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, LDS 751 (U.S. Pat. No. 6,210,885), and the dyes described in dyes described in Georghiou, Photochemistry and Photobiology, 26:59-68, Pergamon Press (1977); Kubota, et al., Biophys. Chem., 6:279-284 (1977); Genest, et al., Nuc. Ac. Res., 13:2603-2615 (1985); Asseline, EMBO J., 3: 795-800 (1984); Richardson, et. al., U.S. Pat. No. 4,257,774; and Letsinger, et. al., U.S. Pat. No. 4,547,569.

“Sybr Green I” refers to N′,N′-dimethyl-N-[4-[(E)-(3-methyl-1,3-benzothiazol-2-ylidene)methyl]-1-phenylquinolin-1-ium-2-yl]-N-propylpropane-1,3-diamine.

### DETAILED DESCRIPTION OF THE INVENTION

I. Introduction

The molecular detection of nucleotide sequences is often limited by the specificity of the assay. For example, detection reagents can bind target nucleotides and also cross-react with non target nucleotides. Such cross-reactivity presents difficulties during differential detection of two nucleic acids simultaneously. Additionally, when one nucleic acid of interest is very similar in sequence to another nucleic acid of interest, it can be difficult to avoid cross reactivity.

Digital methods, in which a sample is partitioned into a set of small mixture partitions and nucleotides are subsequently detected, can also be confounded by detection reagents that cross react. Moreover, when one nucleic acid of interest is common and present at a significantly higher (e.g., 2, 3, 4, 5, 6, 7, 10, 15, 20, 25, 30, 50, 100, 200, 400, 500, 1000, 10,000-fold or higher) concentration than another rare nucleotide to be detected, it can be difficult to distinguish between a partition that contains both the common and the rare nucleotide and a partition that only contains the common nucleotide. For example, to ensure an adequate number of partitions that contain a rare nucleotide, partitions can be loaded with sample at a high concentration of nucleotides per partition. Many of the resulting partitions can contain a high concentration of the common sequence and few or no nucleotides containing the rare sequence.

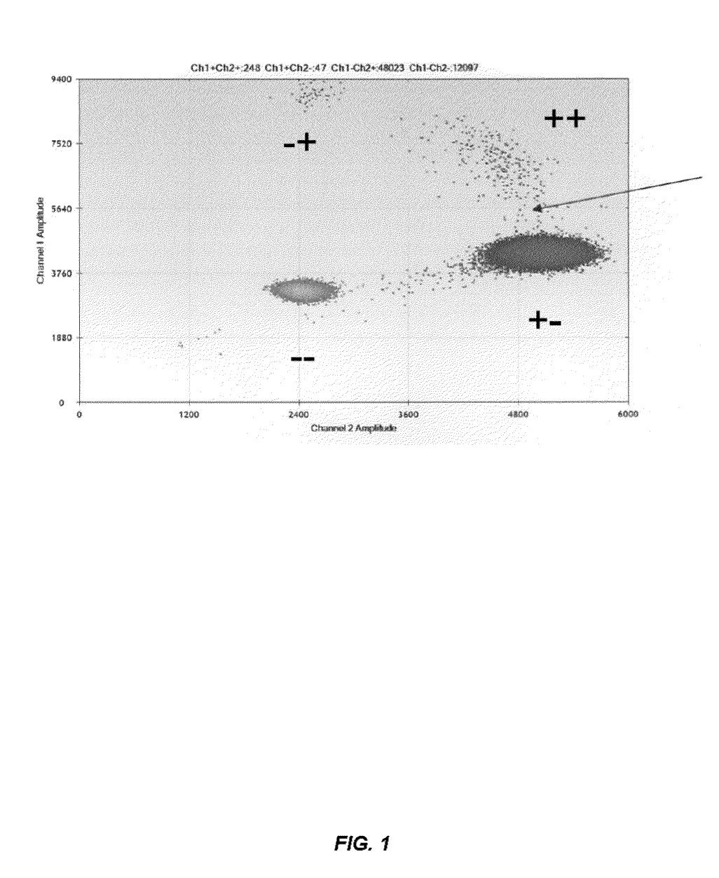

In some cases, cross reactivity of detection reagents and/or large differences in the concentration of two target nucleotides can result in a strong signal in one detection channel corresponding to the common sequence and a weak signal in a second detection channel corresponding to the rare sequence. Additionally, partition clusters containing both the common, e.g. wild-type, sequence and the rare sequence (++) may spread into a diffuse cloud that touches the common, e.g. wild-type, only partitions (+−). This can lead to difficulty in distinguishing partitions that contain only the common sequence from partitions that contain both the common and the rare sequence as shown in FIG. 1. Specifically, partitions clustered near the arrow in FIG. 1 can be difficult to definitely categorize as ++ or +−.

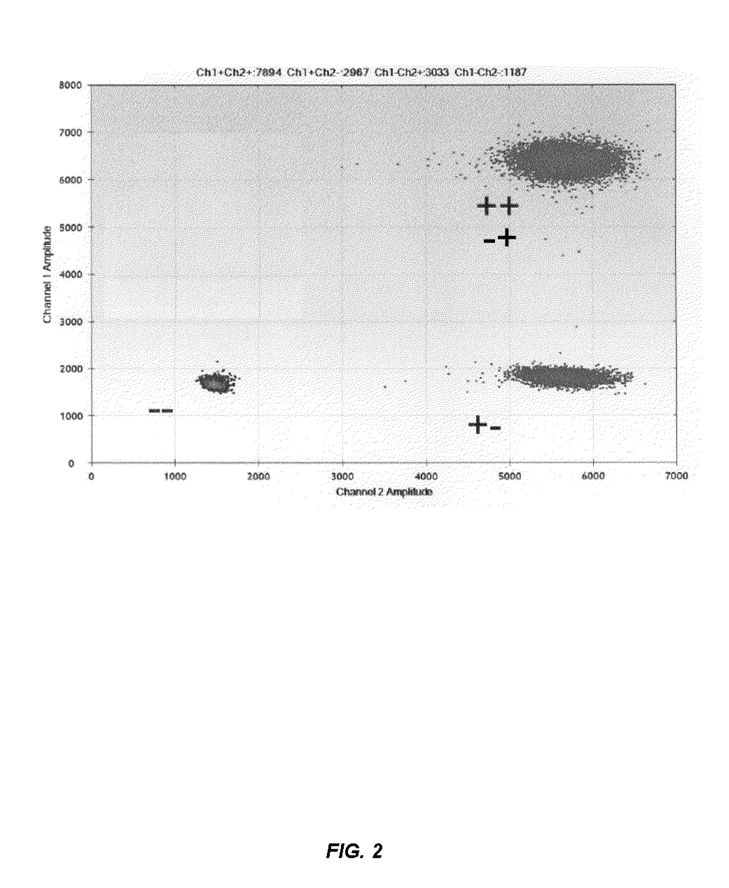

Methods for simultaneous detection or quantification multiple nucleic acids of interest that include a partitioning step can be improved by assaying the partition(s) with one sequence specific detection reagent and one non-specific detection reagent. Such methods can provide the ability to measure the presence or absence of the target nucleic acid corresponding to the sequence specific detection reagent in each partition and the presence or absence of total nucleic acid (e.g., total nucleic acid, total amplified nucleic acid, total reverse transcribed nucleic acid, total DNA, or total double stranded nucleic acid) in each partition, as depicted in FIG. 2. The number of partitions containing the target nucleic acid can correspond to the number of partitions that contain a rare species, such as a mutation, a sequence variant, or a polymorphism. Additionally, the number of partitions containing total nucleic acid can correspond to the number of partitions that contain the rare species plus the number of partitions that contain a common species (e.g., the number of partitions that contain a nucleic acid with a mutant sequence, a wild-type nucleic acid, or both). In some embodiments, the relative proportion of the rare species can then be computed by dividing the number of partitions in which the sequence specific detection reagent detects the presence of a target nucleic acid by the number of partitions in which the non-specific detection reagent detects the presence of nucleic acid.

For example, after digital detection of droplets with a sequence specific detection reagent that detects a rare sequence variant, and a non-specific detection reagent that detects nucleic acid in general (e.g., an intercalating dye), the percentage of droplets containing the rare variant can be calculated as % v=[v]/([wt]+[v]). In this case, v stands for the sequence variant (e.g. a mutation, or a polymorphism, such as a single nucleotide polymorphism (SNP)), wt stands for wild-type, and brackets corresponds to concentration or the relative number of droplets that contain the bracketed species. For example, [v] can correspond to the number of droplets in which the sequence specific detection reagent detects a variant, and ([wt]+[v]) can correspond to the number of droplets in which the non-specific detection reagent detects nucleic acid.

In some embodiments, methods, compositions, and kits are provided herein for quantifying the relative proportion of rare nucleic acids. Such methods, compositions and kits can be useful for diagnosing disease or determining the abundance of a target cell, such as a cancer cell.

II. Compositions

A. Samples

The methods and compositions described herein can be used to detect nucleic acids in any type of sample. In some embodiments, the sample is a biological sample. Biological samples can be obtained from any biological organism, e.g., an animal, plant, fungus, bacteria, or any other organism. In some embodiments, the biological sample is from an animal, e.g., a mammal (e.g., a human or a non-human primate, a cow, horse, pig, sheep, cat, dog, mouse, or rate), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, transformed cells, stem cells; stool; urine; etc.

The sample can contain nucleic acids. In some embodiments, the sample contains target nucleic acids to be detected by the sequence specific detection reagent. In some cases, the sample does not contain target nucleic acids to be detected by the sequence specific detection reagent. In some cases, the sample is suspected of containing target nucleic acids to be detected by the sequence specific detection reagent. In some cases, the sample contains a mixture of target and non-target nucleic acids.

The sample can be prepared to improve the efficient identification of a target nucleic acid and/or total nucleic acid. For example, the sample can be purified, fragmented, fractionated, homogenized, or sonicated. In some embodiments, nucleic acids, or a sub-fraction containing nucleic acids, can be extracted or isolated from a sample (e.g., a biological sample). In some embodiments, the sample is enriched for the presence of the one or more nucleic acids or target nucleic acids. In some embodiments, the nucleic acids or target nucleic acids are enriched in the sample by an affinity method, e.g., immunoaffinity enrichment, or by hybridization. For example, the sample can be enriched for target nucleic acids by immunoaffinity, centrifugation, or other methods known in the art.

In some embodiments, target nucleic acids are enriched in the sample using size selection (e.g., removing very small fragments or molecules or very long fragments or molecules). In other embodiments, the sample is enriched for RNA molecules by selecting for the poly-A tail of eukaryotic messenger RNA. For example, the sample can be passed over an oligo-dT column, and poly-A enriched RNA can be eluted for further analysis.

B. Sequence Specific Detection Reagents

A sequence specific detection reagent suitable for use according to the methods described herein is any molecule that specifically interacts with or specifically binds to a nucleic acid of interest. As such, sequence specific detection reagents of the present invention can be used to detect the presence or absence of the nucleic acid sequence to which it binds. In some cases, the sequence specific detection reagent can discriminate between nucleic acids that differ by e.g., 1, 2, 3, 4, 5, 6, 7, 8, 9, 10, 11, 12, 13, 14, or 15 nucleotides.

For example, in some cases, a sequence specific detection reagent can be used to detect a target nucleic acid sequence and does not, or does not substantially, detect or cross-react with nucleic acids that differ by e.g., 1, 2, 3, 4, 5, 6, 7, 8, 9, 10, 11, 12, 13, 14, or 15 nucleotides. In some embodiments, the sequence specific detection reagent can specifically bind to, or detect, a sequence variant, mutation, or a polymorphism. In some cases, the sequence specific detection reagent can bind to, or detect, a rare sequence. For example, in some cases, the sequence specific detection reagent binds to, or detects, a rare nucleic acid sequence variant that is present in the sample in less than about 10%, 5%, 1%, 0.5%, 0.1%, 0.05%, 0.01%, 0.005%, 0.001%, 0.0005%, 0.00001%, or fewer of the partitions that contain wild-type sequence. In some cases, the sequence specific detection reagent binds to, or detects, a rare nucleic acid sequence variant that is present in the sample in less than about 10%, 5%, 1%, 0.5%, 0.1%, 0.05%, 0.01%, 0.005%, 0.001%, 0.0005%, 0.00001%, or fewer of the partitions that contain wild-type sequence, but does not substantially bind or detect the wild-type sequence.

In some embodiments, the sample is incubated with a sequence specific detection reagent prior to partitioning the sample. In some embodiments, the sample is incubated with a sequence specific detection reagent after partitioning the sample. In some embodiments, the sequence specific detection reagent is present in a mixture. The mixture containing the sequence specific detection reagent can include one or more buffers (e.g., aqueous buffers), one or more additives (e.g., blocking agents or biopreservatives), one or more amplification reagents (e.g. nucleotides, primers, or polymerases), or one or more non-specific detection reagents.

In some embodiments, two or more sequence specific detection reagents can specifically bind to the same target (e.g., at distinct locations or sequences on the same target), if present. For example, each of the two or more sequence specific detection reagents can bind to a different region of the same gene. In some embodiments, two or more sequence specific detection reagents are designed to specifically bind to different target nucleic acids, if present. For example, one sequence specific detection reagent can bind to, or detect, a gene or other nucleotide sequence of interest, such as a sequence variant, a mutation or a polymorphism, and another sequence specific detection reagent can bind to a wild-type sequence or to a control sequence. In some embodiments, the sample is incubated with the two or more sequence specific detection reagents (e.g., in a mixture with the two or more sequence specific detection reagents) under conditions suitable for specifically binding the two or more sequence specific detection reagents to the one or more targets, thereby binding to the one or more target nucleic acids.

In some embodiments, 2, 3, 4, 5, or more sequence specific detection reagents are the same type of molecule (e.g., all nucleic acids). In some embodiments, at least two of the 2, 3, 4, 5 or more sequence specific detection reagents are the same type of molecule (e.g., at least two are nucleic acids). In some embodiments, the 2, 3, 4, 5, or more sequence specific detection reagents are different types of molecules (e.g., an antibody and a nucleic acid).

In some embodiments, the sequence specific detection reagent is a peptide, polypeptide, or protein. As used herein, the terms “peptide,” “polypeptide,” and “protein” interchangeably refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. In some embodiments, the sequence specific detection reagent is an antibody. As used herein, “antibody” refers to a polypeptide of the immunoglobulin family or a polypeptide comprising fragments of an immunoglobulin that is capable of noncovalently, reversibly, and in a specific manner binding a corresponding antigen.

The term antibody also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). Methods for the preparation of antibodies are known in the art; see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985). In some embodiments, the sequence specific detection reagent is a non-antibody protein scaffold. As used herein, a “non-antibody protein scaffold” refers to a non-immunogenic polypeptide that is capable of binding to an identification signature with high specificity. In some embodiments, the protein scaffold has a structure derived from protein A, a lipocalin, a fibronectin domain, an ankyrin consensus repeat domain, or thioredoxin. Methods of preparing non-antibody scaffolds are known in the art; see, e.g., Binz and Pluckthun, Curr Opin Biotechnol 16:459-469 (2005); Koide et al., J Mol Biol 415:393-405 (2012); and Gilbreth and Koide, Curr Opin Struct Biol 22:413-420 (2012).

In some embodiments, the sequence specific detection reagent is a nucleic acid. As used herein, the terms “nucleic acid” and “polynucleotide” interchangeably refer to deoxyribonucleotides or ribonucleotides and polymers thereof in either single or double-stranded form. Examples of nucleic acid based detection reagents are described in Juskowiak, Anal Bioanal Chem. 2011 March; 399(9): 3157-3176, herein incorporated by reference. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Methods of synthesizing polynucleotides are known in the art. See, e.g., Carruthers et al., Cold Spring Harbor Symp. Quant. Biol. 47:411-418 (1982), and Adams et al., J. Am. Chem. Soc. 105:661 (1983). In some embodiments, the sequence specific detection reagent is an oligonucleotide probe that hybridizes to a nucleic acid or sequence of interest. In some embodiments, an oligonucleotide probe is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100, 200, 300, 400, 500, 600, 700, 800, 900, 1000, 1500, 2000, 2500, 3000, 3500, 4000, 4500, 5000, or more nucleotides in length.

In some cases, a single mismatch between the sequence to be detected by the sequence specific detection reagent and the sequence on a target or non-target nucleic acid can result in a decrease in the melting temperature of the interaction between the detection reagent and the target or non-target nucleic acid of about 1, 2, 3, 4, 5, 6, 7, 8, 9, 10, 11, 12, 15, 18, 20, or about 20° C. In some cases, additional mismatches can result in larger decreases in the melting temperature.

In some embodiments, the sequence specific detection reagent is a linear oligonucleotide probe. For example, the sequence specific detection reagent can contain a linear sequence of ribonucleotides, deoxyribonucleotides, nucleotide analogues, or combinations thereof that hybridizes with a nucleic acid of interest. In some cases, linear oligonucleotide probes may contain a label, or a barcode or additional nucleic acid sequence, e.g., for amplification or detection. In some cases, the sequence specific detection reagent contains two oligonucleotides that bind to a nucleic acid at adjacent positions. For example, two probes that bind within 1, 2, 3, 4, 5, 6, 7, 8, 9, 10, 11, 12, 13, 14, 15, 16, 17, 18 19, or about 20 nucleotides. In some cases, one of the probes is labeled with a donor molecular and the other adjacent probe is labeled with an acceptor molecule. Excitation of the donor molecule can cause fluorescence energy transfer to the adjacent acceptor molecule if both probes are bound to a template nucleic acid within a distance of less than about 1, 2, 3, 4, 5, 6, 7, 8, 9, 10, 11, 12, 13, 14, 15, 16, 17, 18 19, or about 20 nucleotides.

In some cases, the linear probe is a hydrolysis probe. For example, a dual-labeled fluorogenic oligonucleotide probe frequently referred to in the literature as a “TaqMan” probe. A sequence specific hydrolysis probe can contain a short (e.g., approximately 20-25 bases in length) polynucleotide that is labeled with two different fluorescent dyes. In some cases, the 5′ terminus of the probe can be attached to a reporter dye and the 3′ terminus attached to a quenching moiety. In other cases, the dyes can be attached at other locations on the probe. The probe can be designed to have at least substantial sequence complementarity with the probe-binding site on the target nucleic acid. Upstream and downstream PCR primers that bind to regions that flank the probe binding site can also be included in the reaction mixture. When the fluorogenic probe is intact, energy transfer between the fluorophore and quencher moiety occurs and quenches emission from the fluorophore. During the extension phase of PCR, the probe is cleaved, e.g., by the 5′ nuclease activity of a nucleic acid polymerase such as Taq polymerase, or by a separately provided nuclease activity that cleaves bound probe, thereby separating the fluorophore and quencher moieties. This results in an increase of reporter emission intensity that can be measured by an appropriate detector.

Alternatively, the sequence specific detection reagent can be a structured probe. Structured probes (e.g., “molecular beacons” or “scorpion probes”) provide another method of detecting a nucleic acid. With molecular beacons, a change in conformation of the probe as it hybridizes to a complementary region of the target nucleic acid results in the formation of a detectable signal. In addition to the target-specific portion, the molecular beacon includes additional sections, generally one section at the 5′ end and another section at the 3′ end, that are complementary to each other. One end section is typically attached to a reporter dye and the other end section is usually attached to a quencher dye. In solution, the two end sections can hybridize with each other to form a stem loop structure. In this conformation, the reporter dye and quencher are in sufficiently close proximity that fluorescence from the reporter dye is effectively quenched by the quencher. Hybridized molecular beacon, in contrast, results in a linearized conformation in which the extent of quenching is decreased. Thus, by monitoring emission changes for the reporter dye, it is possible to detect a nucleic acid. Probes of this type and methods of their use is described further, for example, by Piatek, A. S., et al., Nat. Biotechnol. 16:359-63 (1998); Tyagi, S. and Kramer, F. R., Nature Biotechnology 14:303-308 (1996); and Tyagi, S. et al., Nat. Biotechnol. 16:49-53 (1998).

Scorpion probes generally consist of a single stranded dual labeled fluorescent probe held in a hairpin loop conformation of approximately 20 to 25 nucleotides by complementary stem sequences of approximately 4 to 6 nucleotides on both ends of the probe. The probe contains a 5′ end reporter dye and an internal quencher dye directly linked to the 5′ end of a polymerase primer via a blocker. The blocker prevents polymerase enzymes from extending the primer. The close proximity of the reporter dye to the quencher dye causes the quenching of the reporter's natural fluorescence. During a polymerase reaction, the polymerase extends the primer and synthesizes the complementary strand of the specific target sequence. Denaturation and renaturation unfolds the hairpin loop, and the loop region hybridizes to the newly synthesized target sequence intra-molecularly. This increases the distance between the quencher and the reporter dye leading to an increase in fluorescence.

In some embodiments, the sequence specific detection reagent is an aptamer. An “aptamer,” as used herein, refers to a DNA or RNA molecule that has a specific binding affinity for an identification signature, such as a protein or nucleic acid. In some embodiments, aptamers are selected from random pools based on their ability to bind other molecules with high affinity specificity based on non-Watson and Crick interactions with the target molecule (see, e.g., Cox and Ellington, Bioorg. Med. Chem. 9:2525-2531 (2001); Lee et al., Nuc. Acids Res. 32:D95-D100 (2004)). For example, aptamers can be selected using a selection process known as Systematic Evolution of Ligands by Exponential Enrichment (SELEX). See, e.g., Gold et al., U.S. Pat. No. 5,270,163. Aptamers can be selected which bind, for example, nucleic acids, proteins, small organic compounds, vitamins, or inorganic compounds.

In some embodiments, the sequence specific detection reagent is a nucleic acid primer or a set of nucleic acid primers. For example, the sequence specific detection reagent can be a nucleic acid primer designed to hybridize to a target molecule and prime a polymerase reaction. In some cases, the primer is a primer for first and/or second strand DNA synthesis from an RNA template. In some cases, the primer is a primer for generation of double-stranded nucleic acid, such as double stranded DNA. In some cases, the primer is a PCR primer or a primer for other nucleic acid amplification techniques known in the art, including but not limited to the ligase chain reaction (LCR), the transcription based amplification system (TAS), nucleic acid sequence-based amplification (NASBA), strand displacement amplification (SDA), rolling circle amplification (RCA), hyper-branched RCA (HRCA), and thermophilic helicase-dependent DNA amplification (tHDA).

C. Non-Specific Detection Reagents

Non-specific detection reagents as described herein include any dye that is suitable for detecting nucleic acid. In some embodiments, the non-specific detection reagent is a dye that binds to nucleic acid. In some cases, the dye is a fluorescent dye that binds to nucleic acid. In some cases, the fluorescent dye increases in fluorescence upon binding to nucleic acid. In some cases, the non-specific detection reagent is a dye that intercalates double stranded nucleic acid such as double stranded DNA, double stranded RNA, or RNA:DNA hybrids. Intercalating dyes include any intercalating dye suitable for use in detecting double stranded nucleic acid. Such intercalating dyes include, e.g., 9-aminoacridine, ethidium bromide, a phenanthridine dye, EvaGreen, PICO GREEN (P-7581, Molecular Probes), EB (E-8751, Sigma), propidium iodide (P-4170, Sigma), Acridine orange (A-6014, Sigma), thiazole orange, oxazole yellow, 7-aminoactinomycin D (A-1310, Molecular Probes), cyanine dyes (e.g., TOTO, YOYO, BOBO, and POPO), SYTO, SYBR Green I (U.S. Pat. No. 5,436,134: N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR Green II (U.S. Pat. No. 5,658,751), SYBR DX, OliGreen, CyQuant GR, SYTOX Green, SYTO9, SYTO10, SYTO17, SYBR14, FUN-1, DEAD Red, Hexidium Iodide, ethidium bromide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, LDS 751 (U.S. Pat. No. 6,210,885), and the dyes described in dyes described in Georghiou, Photochemistry and Photobiology, 26:59-68, Pergamon Press (1977); Kubota, et al., Biophys. Chem., 6:279-284 (1977); Genest, et al., Nuc. Ac. Res., 13:2603-2615 (1985); Asseline, EMBO J., 3: 795-800 (1984); Richardson, et. al., U.S. Pat. No. 4,257,774; and Letsinger, et. al., U.S. Pat. No. 4,547,569.

In some embodiments, the non-specific detection reagent is a non-specific nucleic acid binding agent conjugated to a detectable label. For example, the non-specific detection reagent can be an intercalating agent conjugated to a detectable label. In some cases, the non-specific detection reagent can be a protein conjugated to a detectable label. Exemplary proteins capable of binding non-specifically to nucleic acid include single stranded DNA binding protein, and histones.

In some embodiments, the non-specific detection reagent is an oligonucleotide. For example, an oligonucleotide conjugated to a detectable label. In some embodiments, the nucleic acid non-specific detection reagent hybridizes to a universal hybridization sequence. In some cases, the universal hybridization sequence has been incorporated into nucleic acids in the sample by amplification, ligation, or polymerization. For example, the nucleic acids in the sample can be amplified by random primers which contain a hybridization sequence.

In some embodiments, the non-specific detection reagent is a generated during polymerization. For example, during amplification or first or second strand synthesis from an RNA template. In some cases, the non-specific detection reagent is a labeled nucleotide (e.g., a nucleotide labeled with biotin, radioisotope, fluorophore, etc.) that is incorporated into nucleic acids in the sample by amplification, ligation, or polymerization.

D. Detectable Labels

The detection reagents described herein can be detected by detecting a label that is linked to each of the reagents. The label can be linked directly to the detection reagent (e.g., by a covalent bond) or the attachment can be indirect (e.g., using a chelator or linker molecule). The terms “label” and “detectable label” are used synonymously herein. In some embodiments, each label (e.g., a first label linked to a first detection reagent, a second label linked to a second detection reagent, etc.) generates a detectable signal and the signals (e.g., a first signal generated by the first label, a second signal generated by the second label, etc.) are distinguishable. In some embodiments, the two or more labels comprise the same type of agent (e.g., a first label that is a first fluorescent agent and a second label that is a second fluorescent agent). In some embodiments, the two or more labels (e.g., the first label, second label, etc.) combine to produce a detectable signal that is not generated in the absence of one or more of the labels.

Examples of detectable labels include, but are not limited to, biotin/streptavidin labels, nucleic acid (e.g., oligonucleotide) labels, chemically reactive labels, fluorescent labels, enzyme labels, radioactive labels, quantum dots, polymer dots, mass labels, and combinations thereof. In some embodiments, the label can include an optical agent such as a fluorescent agent, phosphorescent agent, chemiluminescent agent, etc. Numerous agents (e.g., dyes, probes, or indicators) are known in the art and can be used in the present invention. (See, e.g., Invitrogen, The Handbook—A Guide to Fluorescent Probes and Labeling Technologies, Tenth Edition (2005)).

Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins (e.g., FITC, 5-carboxyfluorescein, and 6-carboxyfluorescein), benzoporphyrins, squaraines, dipyrrolo pyrimidones, tetracenes, quinolines, pyrazines, corrins, croconiums, acridones, phenanthridines, rhodamines (e.g., TAMRA, TMR, and Rhodamine Red), acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, BODIPY™ and BODIPY™ derivatives, and analogs thereof. In some embodiments, a fluorescent agent is an Alexa Fluor dye. In some embodiments, a fluorescent agent is a polymer dot or a quantum dot. Fluorescent dyes and fluorescent label reagents include those which are commercially available, e.g., from Invitrogen/Molecular Probes (Eugene, Oreg.) and Pierce Biotechnology, Inc. (Rockford, Ill.

In some embodiments, sequence specific detection reagents used for detecting a target molecule are labeled with an optical agent, and each optical agent-labeled detection reagent is detected by detecting a signal generated by the optical agent. In some embodiments, non-specific detection reagents are labeled with an optical agent and detected by detecting the signal generated by the optical agent.

In some embodiments, the label is a radioisotope. Radioisotopes include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In some embodiments, sequence specific detection reagents used for detecting a specific nucleotide sequence are each labeled with a radioisotope (e.g., a first detection reagent labeled with a first radioisotope, a second detection reagent labeled with a second radioisotope, etc.), and each detection reagent that is labeled with a radioisotope is detected by detecting radioactivity generated by the radioisotope. For example, one detection reagent can be labeled with a gamma emitter and one detection reagent can be labeled with a beta emitter. Alternatively, the detection reagents can be labeled with radionuclides that emit the same particle (e.g., alpha, beta, or gamma) at different energies, where the different energies are distinguishable. In some embodiments, sequence specific detection reagents used for detecting a target molecule are labeled with a radioisotope, and each radioisotope-labeled detection reagent is detected by detecting a signal generated by the radioisotope. In some embodiments, non-specific detection reagents are labeled with a radioisotope and detected by detecting the signal generated by the radioisotope. In some cases, the sequence specific detection reagent is labeled with a radioisotope and the non-specific detection reagent is labeled with an optical agent. In other embodiments, the sequence specific detection reagent is labeled with an optical agent and the non-specific detection reagent is labeled with a radioisotope.

In some embodiments, the label is an enzyme, and the detection reagent is detected by detecting a product generated by the enzyme. Examples of suitable enzymes include, but are not limited to, urease, alkaline phosphatase, (horseradish) hydrogen peroxidase (HRP), glucose oxidase, β-galactosidase, luciferase, alkaline phosphatase, and an esterase that hydrolyzes fluorescein diacetate. For example, a horseradish-peroxidase detection system can be used with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, which yields a soluble product readily detectable at 405 nm. A β-galactosidase detection system can be used with the chromogenic substrate o-nitrophenyl-β-D-galactopyranoside (ONPG), which yields a soluble product detectable at 410 nm. A urease detection system can be used with a substrate such as urea-bromocresol purple (Sigma Immunochemicals; St. Louis, Mo.).

In some embodiments sequence specific detection reagents are each labeled with an enzyme (e.g., a first probe labeled with a first enzyme, a second probe labeled with a second enzyme, etc.), and each sequence specific detection reagent that is labeled with an enzyme is detected by detecting a product generated by the enzyme. In some embodiments, all of the sequence specific detection reagents used for detecting a target nucleic acid are labeled with an enzyme, and each enzyme-labeled detection reagent is detected by detecting a product generated by the enzyme. In some embodiments, non-specific detection reagents are labeled with an enzyme and detected by detecting the signal generated by the enzyme. In some cases, the sequence specific detection reagent is labeled with an enzyme and the non-specific detection reagent is labeled with an optical agent or a radioisotope. In other embodiments, the sequence specific detection reagent is labeled with an optical agent or a radioisotope and the non-specific detection reagent is labeled with an enzyme.

In some embodiments, the label is an affinity tag. Examples of suitable affinity tags include, but are not limited to, biotin, peptide tags (e.g., FLAG-tag, HA-tag, His-tag, Myc-tag, S-tag, SBP-tag, Strep-tag), and protein tags (e.g., GST-tag, MBP-tag, GFP-tag).

In some embodiments, the label is a nucleic acid label. Examples of suitable nucleic acid labels include, but are not limited to, oligonucleotide sequences, single-stranded DNA, double-stranded DNA, RNA (e.g., mRNA or miRNA), or DNA-RNA hybrids. In some embodiments, the nucleic acid label is about 10, 15, 20, 25, 30, 35, 40, 45, 50, 60, 70, 80, 90, 100, 150, 200, 250, 300, 350, 400, 450, 500, 600, 700, 800, 900, or 1000 nucleotides in length.

In some embodiments, the label is a nucleic acid barcode. As used herein a “barcode” is a short nucleotide sequence (e.g., at least about 4, 6, 8, 10, or 12, nucleotides long) that uniquely defines a detection reagent molecule, or an nucleic acid bound to a detection reagent. For example, one or more nucleic acids in a partition can be amplified using primers that contain a different barcode sequence in different partitions, thus incorporating a unique barcode into the amplified nucleic acid of the different partitions. Similarly, one or more nucleic acids in a partition can be reverse transcribed using primers that contain a different barcode sequence in different partitions, thus incorporating a unique barcode sequence into the reverse transcribed nucleic acids of the different partitions. Alternatively, or in combination, one or more nucleic acids in a partition can be ligated to a barcode, such that there is a different barcode sequence in the partitions. In some cases, sequence specific detection reagents contain barcodes that are unique to different partitions. In some cases, non-specific detection reagents can contain barcodes that are unique to different partitions. In some cases, both sequence specific and non-specific detection reagents contain barcodes that are unique to different partitions. In such cases, the barcodes may be the same for the specific and the non-specific detection reagent in a given partitions. Alternatively, the barcode may be different for the specific and the non-specific detection reagent in a given partition. Partitions can then be combined, and optionally amplified, without losing track of which partitions contained the nucleic acids. The, presence or absence of nucleic acids containing each barcode can then be counted (e.g. by sequencing) without the necessity of maintaining physical partitions.

The length of the barcode sequence determines how many unique samples can be differentiated. For example, a 4 nucleotide barcode can differentiate 44 or 256 samples or less, a 6 nucleotide barcode can differentiate 4096 different samples or less, and an 8 nucleotide barcode can index 65,536 different samples or less. Additionally, barcodes can be attached to both strands of a double stranded nucleic acid, e.g., through barcoded primers for both first and second strand synthesis from an RNA template, through barcoded primers for amplification of DNA, or through ligation. The use of two distinct barcodes on the two strands increases the number of independent events that can be distinguished.

Alternatively, the same barcode can be attached to the first and second strand of a double stranded nucleic acid. The use of the same barcode, e.g., by incorporating the same barcode in primers for both the first and second strand synthesis from an RNA template, ligation, or by incorporation during amplification of DNA, in each partition can result in identical barcodes on both strands. The dual barcoding can provide a check against subsequent detection errors such as sequencing or amplification errors confounding downstream analysis and allow detection of either or both strands without compromising quantification. The use of barcode technology is well known in the art, see for example Katsuyuki Shiroguchi, et al. Digital RNA sequencing minimizes sequence-dependent bias and amplification noise with optimized single-molecule barcodes, PNAS (2012); and Smith, A M et al. Highly-multiplexed barcode sequencing: an efficient method for parallel analysis of pooled samples, Nucleic Acids Research Can 11, (2010).

In some embodiments, the label is a “click” chemistry moiety. Click chemistry uses simple, robust reactions, such as the copper-catalyzed cycloaddition of azides and alkynes, to create intermolecular linkages. For a review of click chemistry, see Kolb et al., Agnew Chem 40:2004-2021 (2001). In some embodiments, a click chemistry moiety (e.g., an azide or alkyne moiety) can be detected using another detectable label (e.g., a fluorescently labeled, biotinylated, or radiolabeled alkyne or azide moiety).

Techniques for attaching detectable labels to detection reagents are well known. For example, a review of common protein labeling techniques can be found in Biochemical Techniques: Theory and Practice, John F. Robyt and Bernard J. White, Waveland Press, Inc. (1987). Other labeling techniques are reviewed in, e.g., R. Haugland, Excited States of Biopolymers, Steiner ed., Plenum Press (1983); Fluorogenic Probe Design and Synthesis: A Technical Guide, PE Applied Biosystems (1996); and G. T. Herman, Bioconjugate Techniques, Academic Press (1996).

In some embodiments, two or more detection reagent labels (e.g., a first label, second label, etc.) combine to produce a detectable signal that is not generated in the absence of one or more of the labels. For example, in some embodiments, each of the labels is an enzyme, and the activities of the enzymes combine to generate a detectable signal that is indicative of the presence of the labels (and thus, is indicative of each of the detection reagents binding to nucleic acid). Examples of enzymes combining to generate a detectable signal include coupled assays, such as a coupled assay using hexokinase and glucose-6-phosphate dehydrogenase; and a chemiluminescent assay for NAD(P)H coupled to a glucose-6-phosphate dehydrogenase, beta-D-galactosidase, or alkaline phosphatase assay. See, e.g., Macda et al., J Biolumin Chemilumin 1989, 4:140-148.

III. Methods for Detection of Nucleic Acids

In some embodiments, a nucleic acid sequence detection method is provided which comprises:
providing a sample comprising DNA or RNA nucleic acid;
partitioning said sample into a set of mixture partitions;
detecting a presence or absence of a target nucleic acid in the partitions using a sequence specific detection reagent; and
detecting a presence or absence of double-stranded nucleic acids in the partitions using a non-specific detection reagent,
   thereby detecting the ratio of target nucleic acid to total nucleic acid in the partitions.

In some embodiments, a nucleic acid sequence detection method is provided which comprises:
providing a sample comprising DNA or RNA nucleic acid, wherein the DNA or RNA nucleic acid comprises a first target and a second target;
partitioning said sample into a set of mixture partitions; and detecting the first target and the second target in at least one mixture partition with a specific detection reagent that binds to the first target and a nonspecific detection reagent that binds both targets; thereby determining a concentration of the first target and a concentration of the first and second target in the sample.

A. Providing a Sample

The sample can be provided from essentially any biological source. Samples can contain nucleic acids or target nucleic acids. Providing a sample includes obtaining the sample and preparing the sample for the methods provided herein. For example, the sample can be purified, fractionated, enriched or filtered. In some cases, nucleic acids in the sample are amplified, transcribed, reverse transcribed, or ligated. In some cases, the sample is provided and detection reagents (e.g., sequence specific detection reagents, non-specific detection reagents, or a combination thereof) are contacted with the sample prior to the step of partitioning. In some cases, the sample is partitioned and then detection reagents are contacted with the partitioned sample.

B. Partitioning

Samples can be partitioned into a plurality of partitions. Partitions can include any of a number of types of partitions, including solid partitions (e.g., wells or tubes) and fluid partitions (e.g., aqueous droplets within an oil phase). In some embodiments, the partitions are droplets. In some embodiments, the partitions are micro channels. Methods and compositions for partitioning a sample are described, for example, in published patent applications WO 2010/036352, US 2010/0173394, US 2011/0092373, and US 2011/0092376, each of which is incorporated by reference herein in its entirety.

In some cases, samples are partitioned and detection reagents (e.g., probes) are incorporated into the partitioned samples. In other cases, samples are contacted with detection reagents and the sample is then partitioned. In some embodiments, reagents such as probes, primers, buffers, enzymes, substrates, nucleotides, salts, etc. are mixed together prior to partitioning, and then the sample is partitioned. In some cases, the sample is partitioned shortly after mixing reagents together so that substantially all, or the majority, of reactions (e.g., reverse transcription, DNA amplification, DNA cleavage, etc.) occur after partitioning. In other cases, the reagents are mixed at a temperature in which reactions proceed slowly, or not at all, the sample is then partitioned, and the reaction temperature is adjusted to allow the reaction to proceed. For example, the reagents can be combined on ice, at less than 5° C., or at 0, 1, 2, 3, 4, 5, 6, 7, 8, 9, 10, 11, 12, 13, 14, 15, 16, 17, 18, 19, 20, 20-25, 25-30, or 30-35° C. or more. In general, one of skill in the art will know how to select a temperature at which the one or more reactions are inhibited. In some cases, a combination of temperature and time are utilized to avoid substantial reaction prior to partitioning.

Additionally, reagents and sample can be mixed using one or more hot start enzymes, such as a hot start reverse transcriptase or a hot start DNA polymerase. Thus, sample and one or more of buffers, salts, nucleotides, probes, labels, enzymes, etc. can be mixed and then partitioned. Subsequently, the reaction catalyzed by the hot start enzyme, can be initiated by heating the mixture partitions to activate the one or more hot-start enzymes.

Additionally, sample and reagents (e.g., one or more of buffers, salts, nucleotides, probes, labels, enzymes, etc.) can be mixed together without one or more reagents necessary to initiate an intended reaction (e.g., reverse transcription or DNA amplification). The mixture can then be partitioned into a set of first mixture partitions and then the one or more essential reagents can be provided by fusing the set of first mixture partitions with a set of second mixture partitions that provide the essential reagent. Alternatively, the essential reagent can be added to the first mixture partitions without forming second mixture partitions. For example, the essential reagent can diffuse into the set of first mixture partition water-in-oil droplets. As another example, the missing reagent can be directed to a set of micro channels which contain the set of first mixture partitions.

In some embodiments, the sample is partitioned into a plurality of droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes.

In some embodiments, the droplet is formed by flowing an oil phase through an aqueous sample comprising nucleic acids to be detected. In some embodiments, the aqueous sample comprising nucleic acids to be detected further comprises a buffered solution and one or more sequence specific detection reagents for detecting the nucleic acids.

The oil phase can comprise a fluorinated base oil which can additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules can behave as bioreactors able to retain their contents through an incubation period. The conversion to microcapsule form can occur upon heating. For example, such conversion can occur at a temperature of greater than about 40°, 50°, 60°, 70°, 80°, 90°, or 95° C. During the heating process, a fluid or mineral oil overlay can be used to prevent evaporation. Excess continuous phase oil can be removed prior to heating. The microcapsules can be resistant to coalescence and/or flocculation across a wide range of thermal and mechanical processing.

Following conversion, the microcapsules can be stored at about −70°, −20°, 0°, 3°, 4°, 5°, 6°, 7°, 8°, 9°, 10°, 15°, 20°, 25°, 30°, 35°, or 40° C. In some embodiments, these capsules are useful for storage or transport of mixture partitions. For example, samples can be collected at one location, partitioned into droplets containing enzymes, buffers, and/or primers, optionally one or more reverse transcription, amplification, or ligation reactions can be performed, the partitions can then be heated to perform microencapsulation, and the microcapsules can be stored or transported for further analysis.

The microcapsule partitions can contain one or more sequence specific or non-specific detection reagents and can resist coalescence, particularly at high temperatures. Accordingly, the capsules can be incubated at a very high density (e.g., number of partitions per unit volume). In some embodiments, greater than 100,000, 500,000, 1,000,000, 1,500,000, 2,000,000, 2,500,000, 5,000,000, or 10,000,000 partitions can be incubated per mL. In some embodiments, the sample-probe incubations occur in a single well, e.g., a well of a microtiter plate, without inter-mixing between partitions. The microcapsules can also contain other components necessary for the incubation.

In some embodiments, the sample is partitioned into at least 500 partitions, at least 1000 partitions, at least 2000 partitions, at least 3000 partitions, at least 4000 partitions, at least 5000 partitions, at least 6000 partitions, at least 7000 partitions, at least 8000 partitions, at least 10,000 partitions, at least 15,000 partitions, at least 20,000 partitions, at least 30,000 partitions, at least 40,000 partitions, at least 50,000 partitions, at least 60,000 partitions, at least 70,000 partitions, at least 80,000 partitions, at least 90,000 partitions, at least 100,000 partitions, at least 200,000 partitions, at least 300,000 partitions, at least 400,000 partitions, at least 500,000 partitions, at least 600,000 partitions, at least 700,000 partitions, at least 800,000 partitions, at least 900,000 partitions, at least 1,000,000 partitions, at least 2,000,000 partitions, at least 3,000,000 partitions, at least 4,000,000 partitions, at least 5,000,000 partitions, at least 10,000,000 partitions, at least 20,000,000 partitions, at least 30,000,000 partitions, at least 40,000,000 partitions, at least 50,000,000 partitions, at least 60,000,000 partitions, at least 70,000,000 partitions, at least 80,000,000 partitions, at least 90,000,000 partitions, at least 100,000,000 partitions, at least 150,000,000 partitions, or at least 200,000,000 partitions.

In some embodiments, the sample is partitioned into a sufficient number of partitions such that at least a majority of partitions have no more than 1 target nucleic acid (e.g., no more than about 0.1, 0.2, 0.3, 0.5, 1, 2, 3, 4, 5, 6, 7, 8, 9, or 10 target nucleic acids). In some cases, the sample is partitioned such that one target nucleic acid is present in a high number of copies per partition, and another target nucleic acid is present at a small number of copies per partition. For example, one target nucleic acid can be a wild-type sequence that is present at about 1, 2, 3, 5, 6, 8, 10, 12, 14, 15, 20, 25, 30, 50 or more copies per partition. The target nucleic acid present at a small number of copies per partition can be a mutation, polymorphism, or a rare sequence variant that is present in less than about 10%, 5%, 1%, 0.5%, 0.1%, 0.05%, 0.01%, 0.005%, 0.001%, 0.0005%, 0.00001%, or fewer of the partitions. In some embodiments, the sample is partitioned into a sufficient number of partitions such that at least a majority of partitions have no more than 5-10 target and/or non-target nucleic acids (e.g., no more than about 0.5, 1, 2, 3, 4, 5, 6, 7, 8, 9, or 10 target and/or non-target nucleic acids). In some embodiments, a majority of the partitions have no more than 5-10 (e.g., no more than about 0.5, 1, 2, 3, 4, 5, 6, 7, 8, 9, or 10) of the nucleic acids to be detected. In some embodiments, on average about 0.001, 0.005, 0.01, 0.05, 0.1, 0.5, 1, 2, 3, 4, or 5 sequence specific detection reagent molecules are present in each partition.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, the standard deviation of droplet volume can be less than about 1 picoliter, 5 picoliters, 10 picoliters, 100 picoliters, 1 nL, or less than about 10 nL. In some cases, the standard deviation of droplet volume can be less than about 10-25% of the average droplet volume. In some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL.

C. Washing

In some embodiments, after a sample is incubated with two or more probes under conditions suitable for specifically binding the sequence specific detection reagent to a specific nucleic acid sequence, and/or the non-specific detection reagent to nucleic acid (e.g., total nucleic acid, total amplified nucleic acid, total reverse transcribed nucleic acid, total DNA, or total double stranded nucleic acid), the sample is washed to remove detection reagents that do not specifically bind to nucleic acid. In some embodiments, a sample is incubated with a first detection reagent, then optionally subjected to wash conditions before incubating the sample with a second detection reagent. In some embodiments, serially incubating a sample with a detection reagent, then optionally subjecting the sample to wash conditions, then incubating a sample with a different detection reagent can be performed for two, three, four, or five detection reagents or more.

The selection of appropriate wash conditions, wash buffers, etc. will vary based upon conditions such as detection reagent, target molecule, etc., and can be determined by a person skilled in the art. For example, in some embodiments, wherein the detection reagent-nucleic acid complex is denser than the detection reagent alone, the sample can be washed by centrifugation to pellet the detection reagent-nucleic acid complex, followed by resuspension in a buffer lacking detection reagent. As another example, in some embodiments, a detection reagent-nucleic acid complex can be separated from unbound detection reagent by passing the sample through a density gradient or other gradient (e.g., separation by charge). As another example, in some embodiments, a detection reagent-nucleic acid complex can be washed by passing the sample through a column (e.g., size exclusion column) to separate the complex from unbound detection reagent. A wash process can be repeated for additional washes as necessary. In some embodiments, the sample is washed before partitioning. In some embodiments, the sample is washed after partitioning. In some embodiments, no intervening wash step is performed after incubation of the sample with the detection reagents and before detection of the detection reagents.

In some embodiments, the sample is maintained at a controlled temperature or range of temperatures before, during, and/or after partitioning the sample. In some embodiments, the sample is maintained at a temperature of about 20°, 25°, 30°, 35°, 40°, 45°, 50°, 55°, 60°, 65°, 70°, 75°, 80°, 85°, 90°, or 95° C. before, during, and/or after partitioning the sample, e.g., at a temperature to allow for amplification of signal generated by one or more labeled probes. In some cases, the sample temperature is cycled before or after partitioning. In some cases, the temperature cycling provides amplification of detection reagents, labels, and/or target nucleic acids.

D. Detection

A detection reagent or a detectable label can be detected using any of a variety of detector devices. Exemplary detection methods include radioactive detection, optical detection (e.g., absorbance, fluorescence, or chemiluminescence), or mass spectral detection. As a non-limiting example, a fluorescent label can be detected using a detector device equipped with a module to generate excitation light that can be absorbed by a fluorophore, as well as a module to detect light emitted by the fluorophore.

In some embodiments, detectable labels in partitioned samples can be detected in bulk. For example, partitioned samples (e.g., droplets) can be combined into one or more wells of a plate, such as a 96-well or 384-well plate, and the signal(s) (e.g., fluorescent signal(s)) can be detected using a plate reader. In some cases, barcodes can be used to maintain partitioning information after the partitions are combined.

In some embodiments, the detector further comprises handling capabilities for the partitioned samples (e.g., droplets), with individual partitioned samples entering the detector, undergoing detection, and then exiting the detector. In some embodiments, partitioned samples (e.g., droplets) can be detected serially while the partitioned samples are flowing. In some embodiments, partitioned samples (e.g., droplets) are arrayed on a surface and a detector moves relative to the surface, detecting signal(s) at each position containing a single partition. Examples of detectors are provided in WO 2010/036352, the contents of which are incorporated herein by reference. In some embodiments, detectable labels in partitioned samples can be detected serially without flowing the partitioned samples (e.g., using a chamber slide).

Following acquisition of fluorescence detection data, a general purpose computer system (referred to herein as a “host computer”) can be used to store and process the data. A computer-executable logic can be employed to perform such functions as subtraction of background signal, assignment of target and/or reference sequences, and quantification of the data. A host computer can be useful for displaying, storing, retrieving, or calculating diagnostic results from the nucleic acid detection; storing, retrieving, or calculating raw data from the nucleic acid detection; or displaying, storing, retrieving, or calculating any sample or patient information useful in the methods of the present invention.

In some embodiments, the host computer, or any other computer may be used to calculate the proportion of sequence variants present in the sample. For example, the proportion of sequence variants (e.g., mutation, polymorphism, etc.) can be calculated by dividing the number of partitions in which a sequence specific detection reagent detects the sequence variant by the number of partitions in which the non-specific detection reagent detects partitions containing nucleic acid (e.g., total nucleic acid, total amplified nucleic acid, total reverse transcribed nucleic acid, total DNA, or total double stranded nucleic acid).

In some cases, the ratio of partitions in which the sequence specific detection reagent detects a target nucleic acid and the non-specific detection reagent detects nucleic acid can be reported. In some cases, the report includes a diagnosis or a probability of one or more diagnoses. In some cases, the report includes a recommended treatment, such as a pharmaceutical or chemotherapeutic agent. In some cases, the report is displayed on the screen of the host computer. The report can also be stored on computer readable media, transmitted, or printed onto human readable media.

The host computer can be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, can be included. Where the host computer is attached to a network, the connections can be provided via any suitable transport media (e.g., wired, optical, and/or wireless media) and any suitable communication protocol (e.g., TCP/IP); the host computer can include suitable networking hardware (e.g., modem, Ethernet card, WiFi card). The host computer can implement any of a variety of operating systems, including UNIX, Linux, Microsoft Windows, MacOS, or any other operating system.

Computer code for implementing aspects of the present invention can be written in a variety of languages, including PERL, C, C++, Java, JavaScript, VBScript, AWK, or any other scripting or programming language that can be executed on the host computer or that can be compiled to execute on the host computer. Code can also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface can be provided, allowing the user to control operations using a pointing device. Thus, the present invention is not limited to any particular user interface.

Scripts or programs incorporating various features of the present invention can be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

All patents, patent applications, and other publications, including GenBank Accession Numbers, cited in this application are incorporated by reference in the entirety for all purposes.

1. A nucleic acid sequence detection method comprising:
providing a sample comprising a DNA or RNA nucleic acid;
partitioning said sample into a set of mixture partitions;
detecting a presence or absence of a target nucleic acid in the partitions using a sequence specific detection reagent; and
detecting a presence or absence of double-stranded nucleic acid in the partitions using a non-specific detection reagent,
thereby detecting the ratio of target nucleic acid to total nucleic acid in the partitions.

2. The method of claim 1, wherein the nucleic acid is amplified before detection.

3. The method of claim 2, wherein the non-specific detection reagent is a labeled nucleoside triphosphate, and the step of detecting the presence or absence of double-stranded nucleic acid comprises washing away unincorporated labeled nucleoside triphosphate after amplification.

4. The method of claim 1, wherein the non-specific detection reagent is an intercalating dye.

5. The method of claim 4, wherein the intercalating dye is selected from the group consisting of EvaGreen, picogreen, ethidium bromide, SYBR Green I, SYBR Gold, Yo-Yo, Yo-Pro, TOTO, BOXTO, and BEBO.

6. The method of claim wherein the non-specific detection reagent is a primer that detects total double-stranded nucleic acid.

7. The method of claim 1, wherein the sequence specific detection reagent is selected from the group consisting of a structured probe and a linear probe.

8. The method of claim 7, wherein the structured probe is selected from the group consisting of a molecular beacon and a scorpion probe.

9. The method of claim 7, wherein the linear probe is selected from the group consisting of a hybridization probe and a hydrolysis probe.

10. The method of claim 1, wherein the nucleic acid is RNA, and the method further comprises reverse transcribing the RNA nucleic acid.

11. The method of claim 1, wherein the method comprises amplifying two or more potential amplicons.

12. The method of claim 11, wherein one of the potential amplicons is present in less than 50%, 40%, 30%, 20%, 10%, 5%, 1%, 0.5%, 0.1%, 0.05%, 0.01%, or fewer of the mixture partitions in which double-stranded nucleic acid is present.

13. The method of claim 11, wherein the sequence specific detection reagent detects one specific amplicon, and the non sequence specific detection reagent detects any amplicon.

14. The method of claim 1, wherein the sequence specific detection reagent detects a sequence variant.

15. The method of claim 14, wherein the sequence variant is a rare sequence variant.

16. The method of claim 15, wherein double-stranded nucleic acid is present in a plurality of mixture partitions, and the rare sequence variant is present in less than about 50%, 40%, 30%, 20%, 10%, 5%, 1%, 0.5%, 0.1%, 0.05%, 0.01%, or fewer mixture partitions.

17. The method of claim 1, wherein the method further comprises determining a total nucleic acid concentration by counting the number of mixture partitions in which the non-specific detection reagent detects nucleic acid.

18. The method of claim 17, the method further comprising determining a target nucleic acid sequence concentration by counting the number of mixture partitions in which the sequence specific detection reagent detects nucleic acid.

19. The method of claim 18, wherein the method further comprises determining a ratio of mixture partitions in which the sequence specific detection reagent detects nucleic acid to mixture partitions in which the non sequence specific detection reagent detects nucleic acid, wherein the ratio represents the proportion of nucleic acids in the sample that comprise the target nucleic acid.

20. The method of claim 19, wherein the method further comprises reporting the ratio.

21. A nucleic acid sequence detection method comprising:
providing a sample comprising a DNA or RNA nucleic acid, wherein the DNA or RNA nucleic acid comprises a first target and a second target;
partitioning said sample into a set of mixture partitions; and
detecting the first target and the second target in at least one mixture partition with a specific detection reagent that binds to the first target and a nonspecific detection reagent that binds both targets; thereby determining a concentration of the first target and a concentration of the first and second target in the sample.

22. The method of claim 21, wherein the method further comprises amplifying the targets in the mixture partitions, wherein detecting comprises detecting the amplification of the first and second target, and wherein the specific detection reagent binds to amplicons representing the first target if present, and the non-specific detection reagent binds to amplicons representing the first target if present and to amplicons representing the second target if present.

23. The method of claim 21, wherein the detecting comprises determining the presence or absence of the first target and determining the presence or absence of the first or second target in the at least one mixture partition.

24. The method of claim 23, wherein the detecting is performed on a plurality of mixture partitions.

25. The method of claim 24, wherein the method further comprises determining a ratio of mixture partitions comprising the first target to mixture partitions comprising the first or the second target.

26. The method of claim 25, wherein the method further comprises reporting the ratio.

27. The method of claim 21, wherein the first target is a mutant or a polymorphism and the second target is a wild-type nucleotide sequence.

28. A composition comprising a mixture partition of less than about 100 nL comprising:
a nucleic acid comprising DNA or RNA;
a non-specific detection reagent; and
a sequence specific detection reagent.

29. The composition of claim 28, further comprising amplification reagents.

30. The composition of claim 28, wherein the non-specific detection reagent is selected from the group consisting of EvaGreen, ethidium bromide, SYBR Green, SYBR Gold, Yo-Yo, Yo-Pro, TOTO, BOXTO, and BEBO.

31. The composition of claim 28, wherein the non-specific detection reagent is a primer that detects total double-stranded nucleic acid.

32. The composition of claim 28, wherein the non-specific detection reagent is a labeled nucleoside triphosphate.

33. The composition of claim 28, wherein the sequence specific detection reagent is selected from the group consisting of a molecular beacon, a scorpion probe, a hybridization probe, and a hydrolysis probe.

34. A set of mixture partitions, wherein a plurality of the mixture partitions comprises the composition of claim 28.

35. The set of claim 34, wherein the set comprises at least about 100, 200, 500, or 1000 mixture partitions.

36. The set of claim 34, wherein a plurality of the mixture partitions comprises double-stranded nucleic acid.

37. The set of claim 36, wherein a majority of the mixture partitions comprising double-stranded nucleic acid do not comprise a target nucleic acid.

38. The set of claim 36, wherein the target nucleic acid is a sequence variant.
